(19) United States
(12) Patent Application Publication
(10) Pub. No.: US 2024/0189502 A1
(43) Pub. Date: Jun. 13, 2024

(54) SYSTEM FOR REGULATING THE CONCENTRATION OF GLUCOSE IN THE BLOOD OF A PERSON AND USE OF SUCH A SYSTEM

(71) Applicant: INREDA DIABETIC B.V., Goor (NL)

(72) Inventors: Robin KOOPS, Goor (NL); Mickael Gilbert Robert BOULAY, Goor (NL); Gijs Benno WESTEN, Goor (NL)

(73) Assignee: INREDA DIABETIC B.V., Goor (NL)

(21) Appl. No.: 18/285,833

(22) PCT Filed: Apr. 5, 2022
(86) PCT No.: PCT/EP2022/059014

§ 371 Date: Oct. 5, 2023

(30) Foreign Application Priority Data
Apr. 7, 2021 (NL) 2027929

(51) Int. Cl. A61M 5/14; A61M 5/168

(52) U.S. Cl. CPC A61M 5/1407; A61M 5/1413; A61M 5/16877; A61M 2205/14

(57) ABSTRACT

A system for regulating the concentration of glucose in the blood of a person includes a device for selectively supplying at least two substances having at least two ports for connecting to a respective transport element, and a controller for controlling the device, at least two infusion sets, each infusion set including an infusion element, and the transport element. The system includes a mischannelling detection system for detecting mischannelling of the infusion element and transport element and/or for detecting mischannelling of the transport element and port. The controller is arranged for controlling the device to not supply the substance and/or to provide a warning signal to the person if mischannelling is detected.

The invention relates to a system for regulating the concentration of glucose in the blood of a person.

Such a system is known per se and may be used by diabetes patients for regulating the concentration of glucose in their blood. An example of such a system can be found in the international patent application WO2007/049961, but the system may be any other type of a system for regulating the concentration of glucose in the blood of a person. For example, the system may be, but not limited thereto, an artificial pancreas, a bionic pancreas, a closed loop glucose control system, a low glucose suspend system, or a hybrid closed loop control system.

The system may in particular comprise:
a device for selectively supplying at least two substances, said device comprising:
   at least two receiving spaces for accommodating at least two reservoirs containing said substances;
   at least two ports for connecting to a respective transport element, and
   a controller for controlling said device such that a certain amount of a said substance is supplied and thereby infused into the body of the person via a respective infusion set,
at least two infusion sets, each infusion set comprising:
   an infusion element comprising an infusion means for infusing a substance into a body of said person, said infusion means for example being a cannula, catheter, needle, or nozzle, said infusion means comprising an inlet end for receiving said substance and an outlet end for infusing said substance into said body, wherein said infusion element comprises a first connector for connecting to a second connector of a transport element of the infusion set, and
   a said transport element comprising a transport means, such as a conduit, tube, hose or the like, a second connector arranged at an outlet end of the transport means that is connectable to a said first connector of a said infusion element such that in a connected condition in which the second connector of the transport element is connected to the first connector of the infusion element a medium through flow connection is provided between the outlet end of the transport means and the inlet end of the infusion means for feeding the substance to the infusion means, and a third connector arranged at an inlet end of the transport means for connecting to a said port such that in a connected condition in which the third connector is connected to a said port a medium through flow connection is provided between a said reservoir accommodated in a said receiving space and the inlet end of the transport means for receiving a said substance from said reservoir.

Said system may alternatively be referred to as a set of components, i.e. said device and said two infusion sets.

It is noted that said infusion set comprising said infusion element and infusion means may alternatively be referred to as an injection set comprising an injection element and injection means.

Said infusion means may either be partly inserted into the body of the person, for example in particular a said cannula, catheter or needle, or may be arranged close to the body and thereby able to infuse or inject the substance into the body, for example in particular a said nozzle.

Said substance may be infused or injected into the body over a longer period of time or instantaneously.

Each said receiving space may accommodate a respective reservoir.

It is known to improve the safety of a said system according to the preamble by providing two different types of infusion sets and/or by said device having two different ports, wherein a first transport element, i.e. of the first type of infusion set, can only connect to a first port of the two ports and a second transport element, i.e. of the second type of infusion set, can only connect to the other, second port, and wherein the first transport element can only connect to a first infusion element, i.e. of the first type of infusion set, and the second transport element can only connect to a second infusion element, i.e. of the second type of infusion set. The safety of the system is hereby improved because the first type of infusion set can only transport one type of substance, for example insulin, and the second type of infusion set can only transport the other type of substance, for example glucagon. This is so-called mischannelling prevention, wherein it is prevented that a transport element is connected to a wrong port and/or infusion element by a user and thereby prevented that any part of the infusion set infuses a wrong type of substance.

For example, the applicant has achieved such mischannelling prevention by simply using two different infusion sets, each having different first, second and third connectors, and wherein the device has two different ports and two different reservoirs, and thereby easily obtaining this mischannelling prevention.

It is now an object of the invention to improve the mischannelling prevention of the known system.

This object is achieved by a system of the type described in the preamble, which system in accordance with the invention comprises a mischannelling detection system for detecting mischannelling of the infusion element and transport element and/or for detecting mischannelling of the transport element and port, and wherein said controller is arranged for controlling said device to not supply a said substance and/or to provide a warning signal to said person if mischannelling is detected.

Such a mischannelling detection system may provide the advantage that although said two infusion sets may be mechanically at least substantially the same, i.e. the hardware is at least substantially the same, and may thereby be produced using the same production method, mischannelling can be detected if a transport element is connected to a wrong port and/or infusion element and the safety of the device may be achieved by said controller being arranged to not infuse a said substance and/or by providing a warning signal to said person if mischannelling is detected. A mischannelling prevention system is thus provided by said system comprising said mischannelling detection system and said controller being arranged to control said device in accordance with said mischannelling detection system, i.e. to not supply a said substance and/or to provide said warning signal if mischannelling is detected. If no mischannelling is detected, i.e. the transport element is connected to a correct port and/or infusion element, said controller may be arranged for controlling said device to supply a said substance in the regular way of that device, i.e. for example based on an input by a said person and/or based on an algorithm calculating the amount and/or timing of the supply of substance.

It is noted that if a transport element is not connected to any port and/or infusion element, that may be detected by said mischannelling detection system as mischannelling and said controller may be arranged to not supply said substance and/or to provide said warning signal. This may be convenient, because it is intended to also not supply a substance if no transport element is connected to the port and/or if no infusion element is connected to the transport element.

Said port may for example be defined by any suitable interface and/or connector for providing a connection between the device and third connector of the transport element. Said port is arranged in the vicinity of said or a respective receiving space, for example near a receiving end of said or a respective receiving space, such that when the or a respective third connector is connected to the or a respective port a medium through flow connection between the reservoir contained in the receiving space and the transport element is established.

Said controller may be any suitable type of controller, such as for example a (micro)processor.

Said device may alternatively be referred to as a pump.

It is noted that said at least two receiving spaces for accommodating a respective one of the at least two reservoirs may have a different shape and/or size with respect to each other, such that each said receiving space is arranged for receiving one type of reservoir only, for example only a glucagon reservoir or insulin reservoir. This way it may be prevented that the reservoirs are accommodated in the wrong receiving space. Alternatively the receiving spaces may be similar in shape and/or size, but a detection system may be provided for detecting if the correct reservoir is accommodated in the correct receiving space. Alternatively it may be prevented in any other suitable way that the reservoirs are accommodated in the wrong receiving space.

In particular said mischannelling detection system may be arranged for detecting mischannelling of the infusion element and transport element and/or for detecting mischannelling of the transport element and port when the first connector of the infusion element is connected to the second connector of the transport element and/or when the third connector of the transport element is connected to the port, respectively.

In other words, because the two infusion sets are substantially physically the same it is possible to connect a transport element to a wrong port and/or a wrong infusion element, i.e. mischannelling, and in such a connected condition the mischannelling detection system is arranged to detect this mischannelling and no substance will be supplied and/or a warning signal may be provided. Because no substance will be supplied and/or a warning signal may be provided, the user may disconnect the transport element from the port and/or infusion element and may connect it to the other port and/or infusion element.

It is noted that alternatively said mischannelling detection system may be defined as a channelling detection system for detecting channelling, i.e. detecting to which port and/or infusion element the transport element is connected, wherein said controller is arranged to control said device to supply a substance if it is detected that the transport element is connected to the correct port and/or infusion element and to not supply a substance and/or to provide a warning signal if it is detected that the transport element is connected to the incorrect port and/or infusion element.

Said mischannelling detection system may be arranged to detect mischannelling in different ways, as will be clear from the exemplary embodiments described below.

In an embodiment of the system according to the invention said mischannelling detection system is arranged for electronically and/or electrically and/or optically detecting mischannelling.

It is noted that detecting said mischannelling may take place purely electronically, electrically, or optically, or by any suitable combination thereof, and may optionally also be combined with mechanically detecting mischannelling.

In an embodiment of the system according to the invention said transport element and/or said infusion element comprises an electronically and/or electrically and/or optically detectable feature.

Said detectable feature may be an integral feature of the transport element and/or infusion element or may be provided as a separate feature or element that is connectable to said transport element and/or infusion element. In this latter example said detectable feature may be reusable for new infusion sets, by disconnecting said feature from a said transport element and/or infusion element and by connecting it to a new transport element and/or infusion element.

In a specific exemplary embodiment of the system according to the invention said infusion element comprises a at least one first memory and/or said transport element comprises at least one second memory and wherein said controller is arranged to read information from the at least one first and/or second memory and is arranged to detect said mischannelling based on the information read from the at least one first and/or second memory.

Said memory or memories may comprise information indicative of the type of infusion set, more in particular on the type of infusion element and/or transport element, such that mischannelling may be detected based on the information read by said controller. The type of infusion set may for example be a glucagon or insulin infusion set.

Alternatively said memory or memories may comprise unique information, which unique information is prior to the first use of the infusion set not yet indicative of the type of infusion set. At the first use the controller may allocate said unique information to a type of infusion set, for example based on the port to which a said infusion set is connected, because each port is located near and/or allocated to a respective receiving space that is able to (correctly) accommodate only one type of reservoir containing either insulin or glucagon and as such said unique information can be allocated to either insulin or glucagon based on to which port a said infusion set is connected. After allocating said infusion sets said mischannelling can be detected. This alternative variant provides the advantage that prior to their first use the infusion sets can be used for either substance, such that no different infusion sets have to be made.

Any or each said memory may be part of a chip, i.e. for example a memory chip, and may be said electronically detectable feature as described above.

Any or each said memory or chip may be provided as an integral feature of the transport element and/or infusion element or may be provided as a separate feature or element that is connectable to said transport element and/or infusion element. In this latter example said memory or chip may be reusable for new infusion sets.

Said controller may communicate via wires or wirelessly with the memory or memories and in particular the chip(s) for reading said information therefrom.

For example, communication with the at least one first and/or second memory or chip may take place via electronical or electrical circuits. In such an embodiment preferably each of the controller, the transport element, and the infusion element may comprise at least one electrical or electronical circuit or part thereof, wherein communication between the circuits for example takes place via electromagnetic coils or other suitable communication and/or transfer means, or wherein said parts form a completed circuit after connecting the transport element to the port and thereby the device and to the infusion element. The electromagnetic coils may be arranged such that after connecting, in particularly after correctly connecting, the transport element to the port and the infusion element, the electromagnetic coils of each pair at each connection are in a position in which information can be exchanged there between, and may in particular be arranged close to each other and/or extend parallel and/or aligned with respect to each other. Checking if the correct infusion set is connected to the correct port may take place based on information to which port of the device a said infusion set is connected, because, as described above, each of the two receiving spaces of the device is able to (correctly) accommodate only one type of reservoir containing either insulin or glucagon and based on the ports that are allocated to a respective receiving space it can thus be checked if the correct infusion set, i.e. having the correct information stored in the first and/or second memory or chip, is connected to the correct port. In other words, an infusion set having information indicative that it is an insulin infusion set must be connected to the insulin port to have a correct channelling, and an infusion set having information indicative that it is an glucagon infusion set must be connected to the glucagon port to have a correct channelling.

In such an embodiment the circuit part of the transport element may comprise conductive wires that extend along the transport means between the two outer ends and/or connectors of the transport element for providing communication between the device and the second connector and/or the infusion element, wherein the wires may for example be incorporated into the material of the transport means or may be arranged at the inner or outer side of the transport means, in such a way that these are insulated from the substance.

Alternatively or additionally, communication between the device and the second connector and/or infusion element may take place wirelessly, i.e. not via any and in this case not provided wires of the transport element, for example using transmitters, RFID, or other suitable means.

The device and/or controller may comprise a receiving means for receiving the information read from the at least one memory or chip.

In another embodiment of the system according to the invention the at least one first memory and/or second memory is a writable memory and wherein said controller is arranged for writing said at least one writable first and/or second memory.

In such an embodiment it is possible to write desired and/or suitable information on the at least one memory or chip, for example when the infusion set is first used.

It is noted that in such an embodiment all memories provided may be such writable memories or a combination of writable and non-writable memories may be provided.

The at least one writable first and/or second memory may be any suitable type of memory, for example a writable memory chip.

Communication between the at least one writable first and/or second memory and the controller for writing the at least one writable first and/or second memory may take place as described above with respect to reading the at least one first and/or second memory.

In an embodiment of the system according to the invention said controller is arranged for writing said at least one writable first and/or second memory with information indicative of the type of substance to be or that was infused by that infusion element and/or transport element, respectively, such that after writing said at least one writable first and/or second memory with said information said mischannelling can be detected by said controller reading said information.

Said information can be any suitable type of information, for example a number indicative of insulin or glucagon.

In such an embodiment the at least one writable first and/or second memory may be a write once (read many) memory or chip, such that after the information indicative of the type of substance is stored therein, that information cannot be rewritten. Alternatively software may be provided that may be arranged such that said information indicative of the substance cannot be rewritten. Such exemplary embodiments may in particularly be advantageous if the at least one first and/or second memory chip is used for only one, single-use infusion element. If said at least one writable first and/or second memory or chip is provided as a separate element and can be reused for new infusion elements, the at least one writable first and/or second memory or chip may be (re)written more than once, for example every time a new infusion element is installed.

Writing the at least one first and/or second memory or chip by the controller may take place based on information to which port of the device a said transport element is connected, because, as described above, each of the two receiving spaces of the device is able to (correctly) accommodate only one type of reservoir containing either insulin or glucagon and the ports are allocated to a respective receiving space. It is therefore possible to write said at least one first and/or second memory or chip with information indicative of the type of substance, i.e. insulin or glucagon, based on information of the port. Alternatively or additionally, writing of the at least one first and/or second memory or chip may be based on information written in the other memory or chip or in a further memory or chip. For example, the at least one second memory or chip may be pre-set with information indicative of the type of substance or may be written with said information based on the port, and the at least one first memory or chip may then be written based on information of the second memory or chip.

In said above described electronical embodiments in which use is made of at least one memory or chip said device, in particular the controller thereof, may comprise a memory for storing the information of the (writable) first and/or second memory or chip.

Any or each of said memories or chips may be passive and for example powered by the device, in particular if said electronical circuits comprising electromagnetic coils are provided or said parts of electronical circuits are provided or if RFID is used for wireless communication. Alternatively, in particular in an embodiment in which the device communicates wirelessly with any or each of said memories or chips, for example via a transmitter or via RFID, a said electronical circuit that comprises a said memory or chip may comprise a power source, such as a battery, for powering that memory or chip.

It is noted that a or each glucose sensor element of the system may comprise a transmitter for transmitting glucose information to the device. Such a glucose sensor element may be combined with the infusion element as one sensor and infusion element and communication between the sensor and infusion element and the device and/or transport element may then take place via the transmitter of the sensor and infusion element. Alternatively the infusion element and glucose sensor element are provided as separate elements, wherein the infusion element may be located near such a said glucose sensor in use of the system, such that the transmitter may provide wireless communication between both the glucose sensor and the infusion element and in particular the at least one first memory or chip thereof and the device. In this case communication between the glucose sensor element and the infusion element may for example take place via electromagnetic coils or in any other suitable way.

In an embodiment of the system according to the invention said transport element and/or said infusion element and/or said device comprises an electronical and/or electrical and/or optical circuit or part thereof, and wherein said mischannelling detection system is arranged to detect mischannelling when said electronical and/or electrical and/or optical circuit is not a closed circuit when the first connector of the infusion element is connected to the second connector of the transport element and/or when the third connector of the transport element is connected to the port.

Such an embodiment may optionally comprise at least one mechanical feature, such as a switch that closes said electronical and/or electrical and/or optical circuit or part thereof.

It is noted that if the first connector of the infusion element is not connected to the second connector of the transport element and/or when the third connector of the transport element is not connected to the port this may also result in said electronical and/or electrical and/or optical circuit or part thereof not being closed, thereby resulting in a mischannelling detection. As described above, this may be convenient because it is not intended to supply substance if no connection is present.

Correct channelling, and thus allowing for a supply of substance, could be detected based on said electronical and/or electrical and/or optical circuit or part thereof being a closed circuit or part thereof.

The invention also relates to the use of a system according to the invention as described above or below in any of the described embodiments and/or having any one or more of the described features, alone or in any suitable combination, said use comprising the steps, to be performed in any suitable order, of:
a) connecting the third connector of each of the transport elements to a said port of the device and/or connecting the second connector of each of the transport elements to a said first connector of a said infusion element,
b) detecting mischannelling of the transport element and port and/or of the infusion element and transport element using said mischannelling detection system,
c) not infusing a said substance and/or providing a warning signal to said person if mischannelling is detected.

Advantages of such a use are similar to the advantages described above with respect to the system according to the invention.

Steps a) and/or b) may optionally be performed as two sub-steps. For example, step a1) may be the step of connecting the third connector of each of the transport elements to a said port of the device and/or step a2) may be the step of connecting the second connector of each of the transport elements to a said first connector of a said infusion element. For example, step b1) may be the step of detecting mischannelling of the transport element and port using said mischannelling detection system and/or step b2) may be the step of detecting mischannelling of the infusion element and transport element using said mischannelling detection system. In such an embodiment step b1) may be performed after step a1) and/or step b2) may be performed after step a2), or steps a1) and a2) may be performed before steps b1) and b2). It will be thus clear for the skilled person that any suitable order may be chosen.

Step c) may be performed after steps b1) and b2) are both performed, or after step b1) is performed and/or after step b2) is performed.

Step c) could have two alternative steps, i.e.:
c1) not infusing a said substance and/or providing a warning signal if mischannelling is detected, or
c2) allowing infusion of a said substance if no mischannelling, i.e. thus a correct channelling, is detected.

It is noted that if a transport element is not connected to a said port and/or infusion element, that may be detected as mischannelling and step c) or c1) may occur as well. This may be convenient, because it is intended to also not supply a substance if no transport element is connected to the port and/or if no infusion element is connected to the transport element.

In an embodiment of use of the invention, step b) is performed by electronically and/or electrically and/or optically and/or mechanically detecting mischannelling.

In an embodiment of use of the invention, step b) is performed by reading information from the at least one first and/or second memory.

In an embodiment of use of the invention, step b) is performed by detecting if said electrical and/or optical circuit is not a closed circuit when the first connector of the infusion element is connected to the second connector of the transport element and/or when the third connector of the transport element is connected to the port.

It is noted that in any of the described embodiments correct channelling can also be detected, and thereby step c2) can be performed. More specifically, correct channelling can be detected electronically and/or electrically and/or optically and/or mechanically, for example by reading information from the first and/or second memory and/or by detecting a closed circuit.

Any further or alternative steps of the use of the system will be apparent based on the description of the system above.

For example, a step of writing the at least one first and/or second writable memory can be foreseen, for example with said desired and/or suitable information and/or said information indicative of the type of substance to be or that was infused by that infusion element and/or transport element, respectively.

The invention(s) will be further elucidated with reference to figures, wherein:

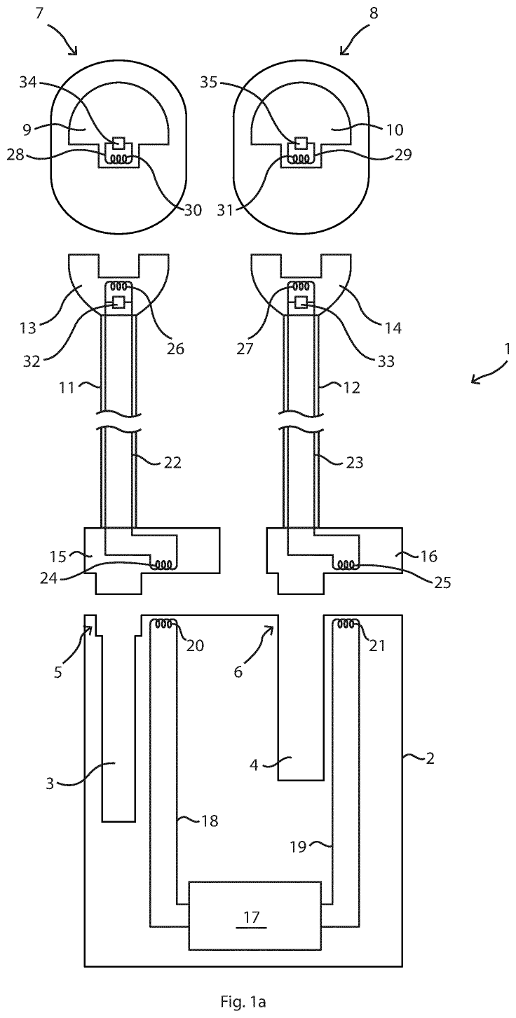

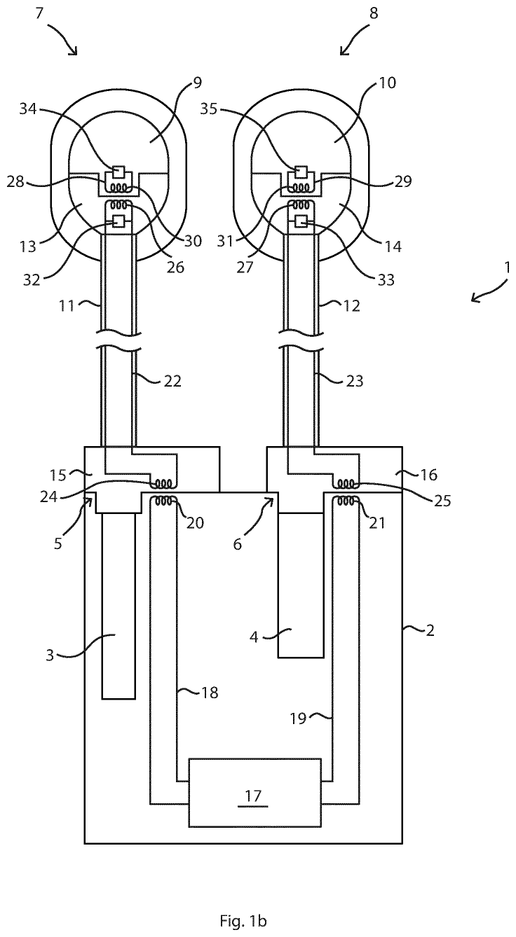

FIGS. 1a and 1b schematically show a first embodiment of the system according to the invention, wherein FIG. 1a shows the infusion set in a disconnected condition and FIG. 1b shows the infusion set in a connected condition:

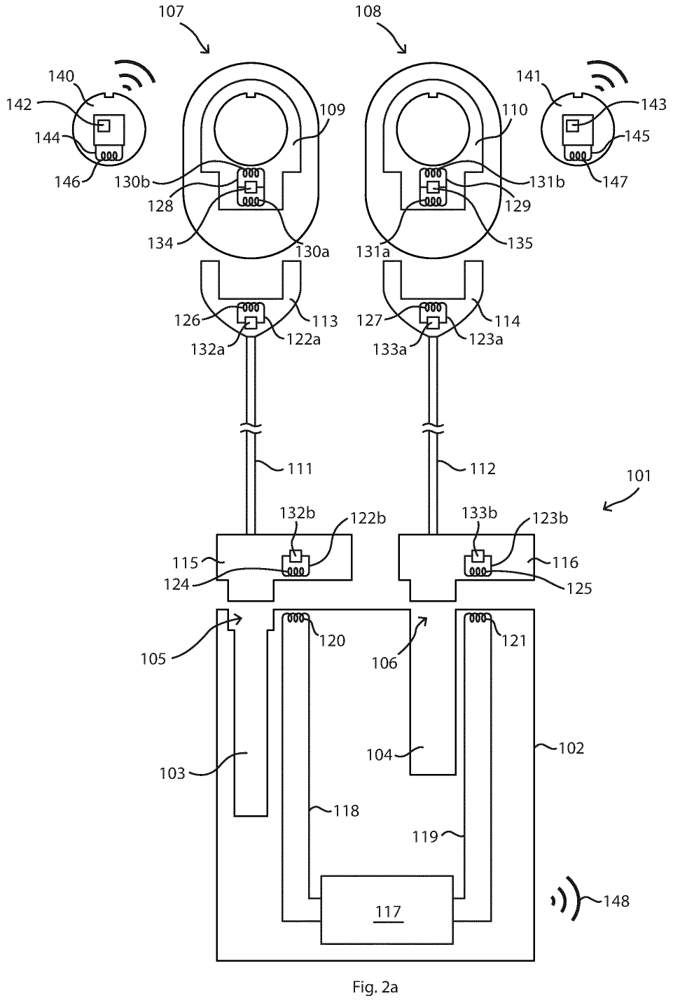

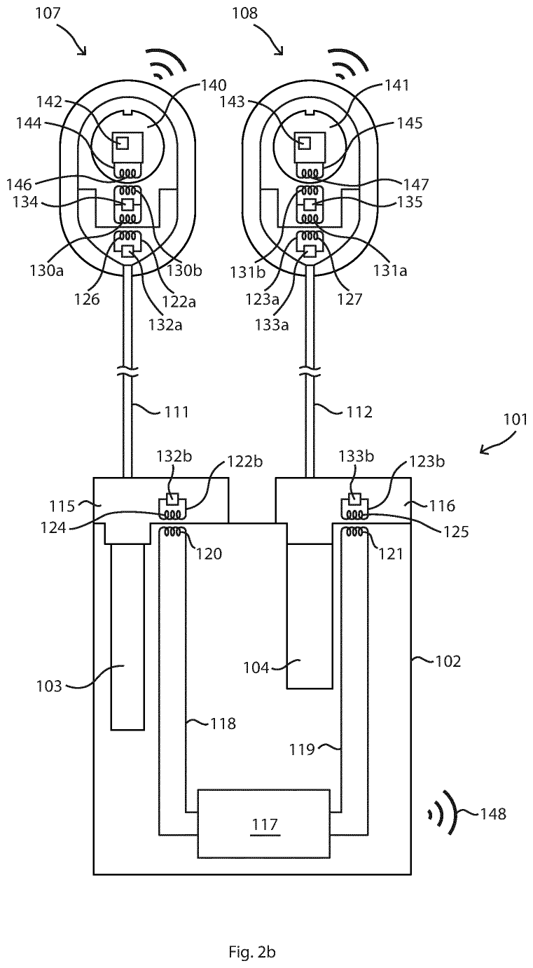

FIGS. 2a and 2b schematically show a second embodiment of the system according to the invention, wherein FIG. 2a shows the infusion set in a disconnected condition and FIG. 2b shows the infusion set in a connected condition:

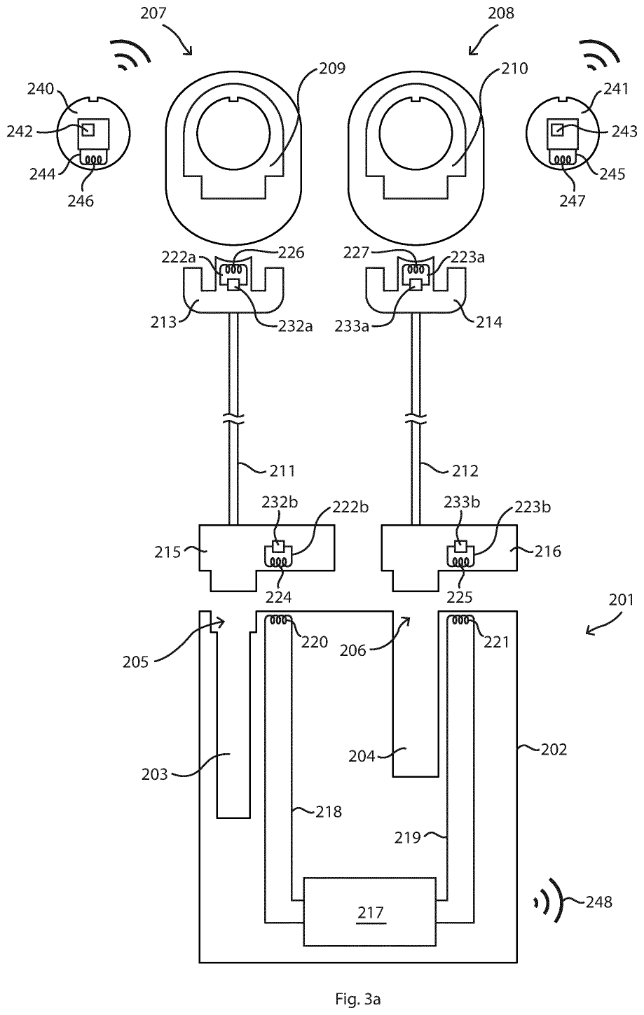

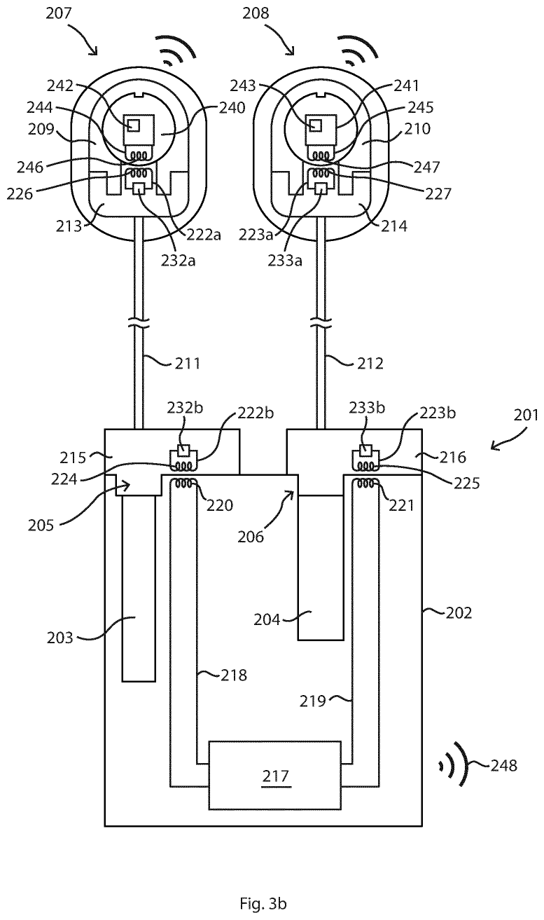

FIGS. 3a and 3b schematically show a third embodiment of the system according to the invention, wherein FIG. 3a shows the infusion set in a disconnected condition and FIG. 3b shows the infusion set in a connected condition:

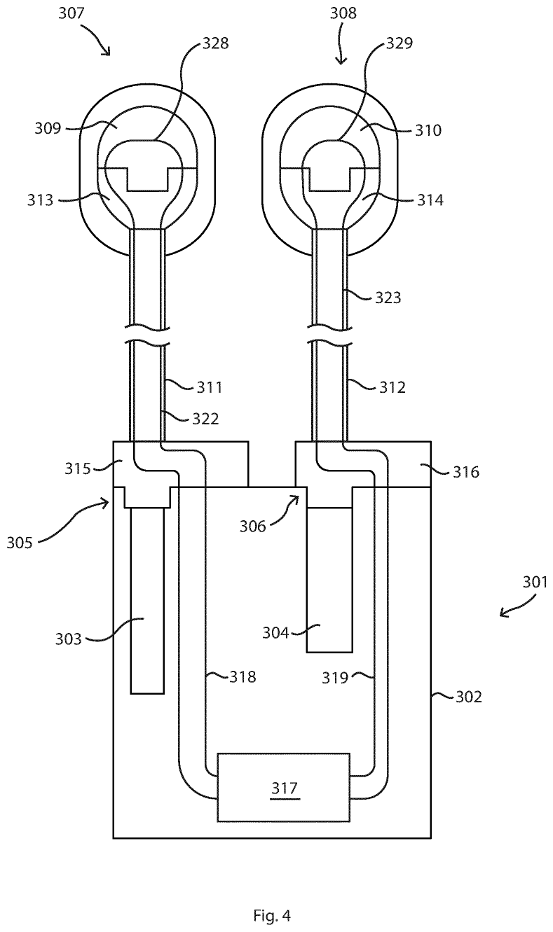

FIG. 4 schematically shows a fourth embodiment of the system according to the invention.

In the figures same or similar features are referred to by same reference numerals, increased by one hundred (100), two hundred (200) and three hundred (300) for the second, third and fourth embodiment, respectively.

For the sake of clarity and/or simplicity features that may be relevant for understanding the invention are shown in the figures, even though such features might not normally be visible in the views as shown. Further, some features of the system may not be shown for the sake of simplicity. The features that are shown may be shown in a simplified and/or enlarged way and/or in any other way different from reality but improving the readability of the figures.

FIGS. 1a and 1b show a system 1 for regulating the concentration of glucose in the blood of a person in two different conditions, as will be described below. Said system 1 comprises a device 2 for selectively supplying in this embodiment two substances, i.e. insulin and glucagon. Said device 2 comprises in this embodiment two receiving spaces 3, 4 for receiving and accommodating respective reservoirs containing said substances. As is shown in FIG. 1, in this embodiment the receiving spaces 3, 4 each have a different length and diameter, wherein the length and diameter are adapted to the type of reservoir to be received therein, such that each receiving space 3, 4 is able to receive only one type of reservoir. In particular, in this embodiment the receiving space 3 is able to receive an insulin reservoir and the receiving space 4 is able to receive a glucagon reservoir, which glucagon reservoir is shorter and thicker as compared to the insulin reservoir. The device 2 further comprises ports 5, 6, via which ports 5, 6 a respective reservoir may be inserted into the respective receiving space 3, 4 and to which an infusion set can be connected.

FIGS. 1a, 1b further show two infusion sets, wherein each infusion set comprises an infusion element 7, 8 comprising an infusion means for infusing a said substance into the body of said person, said infusion means for example being a cannula, catheter, needle, or nozzle, and said infusion means comprising an inlet end for receiving said substance and an outlet end for infusing said substance into said body. Such an infusion means is provided at the back of the infusion element 7, 8 as shown in FIGS. 1a, 1b and therefore not visible in FIGS. 1a, 1b. However, such an infusion element is well known to the skilled person and thus the skilled person would understand how to embody such an infusion means. The infusion elements 7, 8 further each comprise a respective first connector 9, 10.

In this embodiment each infusion set further comprises a respective tube 11, 12 as transport means that is part of a transport element. At an outlet end of the tubes 11, 12 each tube 11, 12 comprises a second connector 13, 14 that is connectable to a said first connector 9, 10 of a said infusion element 7, 8 such that in a connected condition as shown in FIG. 1b in which the second connectors 13, 14 are connected to the first connectors 9, 10 a medium through flow connection is provided between the outlet end of the tubes 11, 12 and the inlet end of the infusion means for feeding the substance to the infusion means. At an inlet end of the tubes 11, 12, which is located at the other end of the tubes 11, 12, said tubes comprise a respective third connector 15, 16 for connecting to a said port 5, 6 such that in a connected condition as shown in FIG. 1b in which a said third connector 15, 16 is connected to a said port 5, 6 a medium through flow connection is provided between a said reservoir contained in a said receiving space 3, 4 and the inlet end of the tubes 11, 12 for receiving a said substance from a said reservoir. A correct channelling is obtained if in the connected condition of FIG. 1b each of the third connectors 15, 16 is connected to the correct respective port 5, 6 and each of the second connectors 13, 14 is connected to the correct respective first connector 9, 10, as will be explained further below.

Said system 1, and in this embodiment in particular the device 2, comprises a controller 17 for controlling said device 2 such that a certain amount of a said substance is supplied and thereby infused into the body of the person via a respective infusion set.

As will be clear from FIGS. 1a, 1b, the first connectors 9, 10, second connectors 13, 14, third connectors 15, 16 and ports 5, 6 are in this embodiment mechanically substantially the same. Therefore it is possible to connect any first connector 9, 10 to any second connector 13, 14 and to connect any third connector 15, 16 to any port 5, 6. This makes the system 1 mechanically relatively simple and/or cheap to manufacture, but it can give risks when the tubes 11, 12 are connected to the wrong port 5, 6, especially if said tube has already been in use and is thus already filled with the other substance, or when the infusion elements 7, 8 are connected to the wrong tube 11, 12. In accordance with the invention the system 1 thus comprises a mischannelling detection system for in this embodiment detecting mischannelling of the tubes 11, 12 with the ports 5, 6 and infusion elements 9, 10, and wherein said controller 17 is arranged for controlling said device 2 to not supply a said substance and/or to provide a warning signal to said person if mischannelling is detected.

In this first embodiment of FIGS. 1a and 1b said mischannelling detection system comprises an electronical mischannelling detection system. The controller has two electrical or electronical circuits 18, 19, wherein each circuit 18, 19 comprises a respective electromagnetic coil 20, 21. The transport elements each have a respective electronical circuit 22, 23 comprising respective wires that extend along the tubes 11, 12 from and including the respective second connectors 13, 14 and third connectors 15, 16, wherein each circuit 22, 23 comprises a respective electromagnetic coil 24, 25 in the respective third connector 15, 16 and a respective electromagnetic coil 26, 27 in the respective second connector 13, 14. The infusion elements 7, 8 and in particular the respective first connectors 9, 10 thereof each have a respective electronical circuit 28, 29, each circuit 28, 29 comprising a respective coil 30, 31. In the connected condition of FIG. 1b the coils 20, 21 are located adjacent the coils 24, 25, and the coils 26, 27 are located adjacent the coils 30, 31 such that communication between the various electronical circuits 18, 22, 28, and 19, 23, 29 can take place via the coils. Each circuit of the infusion elements 7, 8 has a respective first memory, embodied here as first chip 34, 35 and in this case provided as part of the respective first connectors 9, 10, and each circuit 22, 23 of the transport elements has a respective second memory, embodied here as second chip 32, 33 and in this case provided as part of the respective second connectors 13, 14. In particular the chips 32-35 are in this embodiment provided as integral parts of the transport elements and infusion elements, respectively.

The first and second chips may be pre-set, i.e. pre-written with information on the type of substance to be infused with that infusion set. The controller 17 can check if the correct transport element is connected to the correct port 5, 6 by reading the information of the second chips 32, 33 and checking if that information is read, i.e. received, via the correct electronical circuit 18, 19. The controller 17 can further check if the correct infusion element 7, 8 is connected to the transport element by reading the information of the first chips 34, 35 and checking if that information is also read, i.e. received, via the correct electronical circuit 18, 19, wherein communication between the controller 17 and the first chips 34, 35 takes place via the circuits 22, 23 of the transport elements.

Alternatively the first and second chips may be pre-set, i.e. pre-written with information, but said information may not yet be indicative of the type of substance. For example, each chip may comprise unique information, such as a unique number, and upon the first use of the infusion sets the controller 17 may be arranged to allocate said unique information to the type of substance based on via which port said information is received. In further use of the infusion sets the correct channelling or mischannelling can be detected based on this unique and now allocated information. This alternative variant provides the advantage that prior to their first use the infusion sets can be used for either substance.

Alternatively the first and second chips may be writable chips. The controller 17 can write the second chips 32, 33 with information indicative of the type of substance and then the first chips 34, 35 are written based on the informed stored in the second chips 32, 33. Writing said chips directly or indirectly via the controller 17 with said information takes place via the circuits 18, 19, 22, 23, 28, 29 and by knowing to which port 5, 6 the circuits 18, 19 are related. Writing said chips 32, 33, 34, 35 may take place at the first use of a new infusion set. After writing the chips 32, 33, 34, 35 it may not be possible to overwrite the chips, such that after the first use the infusion sets cannot be used for the other substance. After writing the chips the controller 17 can check if the correct transport element is connected to the correct port 5, 6 by reading the information of the second chips 32, 33 and checking if that information is read, i.e. received, via the correct electronical circuit 18, 19. The controller 17 can further check if the correct infusion element 7, 8 is connected to the correct transport element by reading the information of the first chips 34, 35 and checking if that information is also read, i.e. received, via the correct circuit 18, 19, wherein communication between the controller 17 and the first chips 34, 35 takes place via the circuits 22, 23 of the transport elements. This alternative variant provides the advantage that prior to their first use the infusion sets can be used for either substance.

It the controller 17 has detected that the correct transport elements are connected to the correct ports 5,6 and the correct infusion elements 7, 8 are connected to the correct transport elements, said controller 17 may be arranged to supply a said substance in accordance with a normal use of the device, for example upon request by a user inputting to supply a dose or in accordance with an algorithm calculating the amount of substance to be dosed. If the controller 17 has detected that the transport elements are connected to the wrong ports 5,6 and/or the infusion elements 7, 8 are connected to the wrong transport elements, said controller 17 is arranged to not supply a said substance and/or to provide a warning signal.

In this embodiment all circuits may be powered via the device 2.

FIGS. 2a, 2b show a second embodiment of the system 101. Only the differences with the first embodiment of FIGS. 1a and 1b will be described here. For a further description of the second embodiment of FIGS. 2a, 2b the reader is referred to the description of FIGS. 1a and 1b.

The second embodiment of the system 101 of FIGS. 2a, 2b differs in that the infusion elements 107, 108 communicate via a respective transmitter 140, 141 with the controller 117. The transmitters 140, 141 are provided as separate elements that can be releasably connected to the infusion elements 107, 108 and thereby reused for new infusion sets. Each transmitter 140, 141 comprises a respective electrical or electronical circuit 144, 145 which comprise a respective coil 146, 147 and respective third chip 142, 143. If the transmitters 140, 141 are connected to the respective infusion elements 107, 108 as shown in FIG. 2b the transmitters are able to communicate with the electronical circuits 128, 129 of the first connectors 109, 110 via respective coils 130b, 131b and thereby to receive the information from the first chips 134, 135. In this embodiment the electronical circuits 128, 129 of the first connectors 109, 110 each comprise two sets of coils. i.e. the just described coils 130b, 131b for communicating with the respective transmitters 140, 141, and coils 130a, 130b for communicating with respective coils 126, 127 of circuits 122a, 123a of the transport elements, as is similar as described with respect to FIGS. 1a, 1b. This way the transmitters 140, 141 are also able to obtain the information of chips 132a, 133a of the transport elements and to communicate both information wirelessly to the controller 117 by transmitting this information. Because the controller 117 receives the information of chip pairs 132a, 134, and of chip pairs 133a, 129 the controller 117 is able to detect if the infusion element is connected to the correct transport element.

A further difference of this embodiment is that the transport elements each comprise two second chips 132a, 132b, respectively, and 133a, 133b respectively. The first second chips 132a, 133a are comprised in circuits 122a, 123a of the second connectors 113, 114 of the transport elements, and the second second chips 132b, 133b are comprised in circuits 122b, 123b of the third connectors 115, 116 of the transport elements. As described above, the information of the first second chips 132a, 133a can be exchanged with the circuits 128, 129 of the first connectors 109, 110 of the infusion elements 107, 108 via coil pairs 130a, 126 and 131a, 127. The information of the second second chips 132b, 133b can be exchanged with the device 102 via respective coil pairs 124, 120 and 125, 121. The controller 117 is thus arranged to receive the information of the second second chips 132b, 133b via said respective coils 124, 125 and is thereby able to detect if the third connectors 115, 116 are connected to the correct port 105, 106 and thereby if the transport elements are connected to the correct port.

In this embodiment the transmitters 140, 141 comprise a battery or other power source for powering the transmitter, but also the circuits 128, 129 and 122a, 123a of the first and second connectors respectively.

For receiving the information from the transmitters 140, 141 said controller 117 comprises a receiving means, which is schematically illustrated by means of waves 148.

It is noted that the transmitters 140, 141 are shown as separate elements. Alternatively these can be an integral part of the infusion elements 107, 108. Alternatively the transmitters 140, 141 can be part of a glucose sensor (not shown) and may be located near the circuits 128, 129 and thereby able to communicate with the circuits 128, 129, for example via coils as shown before.

As described above with respect to the first embodiment, the first and second chips may be pre-set, i.e. pre-written with information on the type of substance to be infused with that infusion set. In such an embodiment the chips 142, 143 of the transmitters 140, 141 are not required for detecting correct or incorrect channelling, but may optionally be provided and may for example be used for storing the information of the first chips 134, 135 and second chips 132a, 133a, respectively.

Alternatively the first and second chips may be pre-set, i.e. pre-written with said unique information that can be allocated to the type of substance at the first use of the infusion sets or may be writable chips. In such embodiments the transmitters 140, 141 may be dedicated transmitters for either insulin or glucagon and this information may be stored in the chips 142, 143 of the transmitters 140, 141, respectively. The information stored in chips 142, 143 may be used for allocating the pre-set information of the first chips and first second chips or for writing these chips. The second second chips may be written or the pre-set information thereon may be allocated based on to which port the thirds connectors are connected, as described above. After writing the chips or allocating the information stored therein the correct or incorrect channelling can be detected as described above.

It is noted that even though some elements are disclosed to comprise a circuit comprising two coils, only one coil may be provided where applicable. This will be clear for the skilled person.

FIGS. 3a, 3b show a third embodiment of the system 201. Only the differences with the second embodiment of FIGS. 2a and 2b will be described here. For a further description of the third embodiment of FIGS. 3a, 3b the reader is referred to the description of FIGS. 2a and 2b and where relevant 1a, 1b.

The system 201 of FIGS. 3a, 3b differs from the second embodiment of FIGS. 2a, 2b in that the infusion elements 207, 208 and in particular the first connectors 209, 210 thereof do not comprise a circuit with chip. Instead the chips 242, 243 of the transmitters 240, 241 are in this embodiment used as the first chips of the infusion elements 207, 208 after connecting the transmitters 240, 241 to the infusion elements 207, 208. As is best shown in FIG. 3b, after connecting the transmitters 240, 241 to the infusion elements 207, 208 the respective coil pairs 226, 246 and 227, 247 provide for communication between the circuits 222a, 244, and 223a, 245 respectively. This way the transmitters 240, 241 are able to obtain the information from the chips 232a, 233a and this is transmitted to the controller 217 together with the information of the chips 242, 243 of the transmitters 240, 241. The transmitters 240, 241 may be dedicated transmitters for insulin or glucagon and the information stored in their respective chips 242, 243 may thus contain information indicative on the type of substance. By transmitting the information of both chips 232a, 242 and 233a, 243 by means of the transmitter, the controller 217 is able to receive the information and thus able to detect correct or incorrect channelling of the infusion element with the transport element. The detection of the correct channelling or mischannelling of the transport element with the port is performed as described before.

As explained before the second chips may either be pre-set or writable chips.

FIG. 4 shows a fourth embodiment of the system 301 in the connected condition. Only the differences with the first embodiment of FIGS. 1a and 1b will be described here. For a further description of the fourth embodiment of FIG. 4 the reader is referred to the description of FIGS. 1a and 1b.

In the fourth embodiment an electrical or optical mischannelling detection system is used. In this embodiment electrical or optical circuit parts 318, 319, 322, 323, 328, 329 are provided respectively as part of the device 302, transport elements and infusion elements 307, 308. The difference of this embodiment is that the circuit parts do not communicate with each other via coils, but that the circuit parts form a closed circuit in the connected condition of the transport elements with the device 302 and infusion elements 307, 308. As is clear from the figure, the locations where the circuits parts connect to each other at the various interfaces are arranged at different locations at each interface, i.e. at the interface between the ports 305, 306 and third connectors 315, 316, and at the interface between the second connectors 313, 314 and first connectors 309, 310, such that the circuit parts only form a closed circuit if the correct transport elements are connected to the correct ports 305, 306 and the correct infusion elements 307, 308. If the circuit parts 318, 322, 328 and 319, 323, 329 together form a closed circuit the controller 317 is able to transmit and receive an electrical or optical signal, thereby being able to detect the correct channelling. If a signal is transmitted but not received, the controller 317 is able to detect said mischannelling.

It is noted that the invention is not limited to the shown embodiments but also extends to variants within the scope of the appended claims.

1. A system for regulating the concentration of glucose in the blood of a person, said system comprising:
a device for selectively supplying at least two substances, said device comprising:
at least two receiving spaces for accommodating at least two reservoirs containing said substances;
at least two ports for connecting to a respective transport element, and
a controller for controlling said device such that a certain amount of said substance is supplied and thereby infused into the body of the person via a respective infusion set,
at least two infusion sets, each infusion set comprising:
an infusion element comprising an infusion means for infusing a substance into a body of said person, said infusion means comprising an inlet end for receiving said substance and an outlet end for infusing said substance into said body, wherein said infusion element comprises a first connector for connecting to a second connector of a transport element of the infusion set, and
said transport element comprising a transport means, a second connector arranged at an outlet end of the transport means that is connectable to said first connector of said infusion element such that in a connected condition in which the second connector of the transport element is connected to the first connector of the infusion element a medium through flow connection is provided between the outlet end of the transport means and the inlet end of the infusion means for feeding the substance to the infusion means, and a third connector arranged at an inlet end of the transport means for connecting to said port such that in a connected condition in which the third connector is connected to said port a medium through flow connection is provided between said reservoir accommodated in said receiving space and the inlet end of the transport means for receiving said substance from said reservoir,
wherein said system comprises a mischannelling detection system for detecting mischannelling of the infusion element and transport element and/or for detecting mischannelling of the transport element and port, and wherein said controller is arranged for controlling said device to not supply said substance and/or to provide a warning signal to said person if mischannelling is detected.

2. The system according to claim 1, wherein said mischannelling detection system is arranged for detecting mischannelling of the infusion element and transport element and/or for detecting mischannelling of the transport element and port when the first connector of the infusion element is connected to the second connector of the transport element and/or when the third connector of the transport element is connected to the port, respectively.

3. The system according to claim 1, wherein said mischannelling detection system is arranged for electronically and/or electrically and/or optically detecting mischannelling.

4. The system according to claim 1, wherein said transport element and/or said infusion element comprises an electronically and/or electrically and/or optically detectable feature.

5. The system according to claim 1, wherein said infusion element comprises at least one first memory and/or said transport element comprises at least one second memory and wherein said controller is arranged to read information from the at least one first and/or second memory and is arranged to detect said mischannelling based on the information read from the at least one first and/or second memory.

6. The system according to claim 5, wherein the at least one first memory and/or second memory is a writable memory and wherein said controller is arranged for writing said at least one writable first and/or second memory.

7. The system according to claim 6, wherein said controller is arranged for writing said at least one writable first and/or second memory with information indicative of the type of substance to be or that was infused by that infusion element and/or transport element, respectively, such that after writing said at least one writable first and/or second memory with said information said mischannelling can be detected by said controller reading said information.

8. The system according to claim 1, wherein said transport element and/or said infusion element and/or said device comprises an electronical and/or electrical and/or optical circuit or part thereof, and wherein said mischannelling detection system is arranged to detect mischannelling when said electronical and/or electrical and/or optical circuit is not a closed circuit when the first connector of the infusion element is connected to the second connector of the transport element and/or when the third connector of the transport element is connected to the port.

9. A use of a system according to claim 1, said use comprising the steps, to be performed in any order, of:
a) connecting the third connector of each of the transport elements to said port of the device and/or connecting the second connector of each of the transport elements to said first connector of said infusion element,
b) detecting mischannelling of the transport element and port and/or of the infusion element and transport element using said mischannelling detection system, and
c) not infusing said substance and/or providing a warning signal to said person if mischannelling is detected.

10. The use according to claim 9, wherein step b) is performed by electronically and/or electrically and/or optically detecting mischannelling.

11. The use according to claim 9, wherein step b) is performed by reading information from the at least one first and/or second memory.

12. The use according to claim 9, when step b) is performed by detecting if said electronical and/or electrical and/or optical circuit is not a closed circuit when the first connector of the infusion element is connected to the second connector of the transport element and/or when the third connector of the transport element is connected to the port.
